(19) United States
(12) Patent Application Publication
(10) Pub. No.: US 2009/0299160 A1
(43) Pub. Date: Dec. 3, 2009

(54) INFORMATION PROCESSING APPARATUS, INFORMATION PROCESSING METHOD, INFORMATION PROCESSING PROGRAM AND RECORDING MEDIUM

(76) Inventors: Tomohisa MORIDAIRA, Tokyo (JP); Yoshihiro KUROKI, Kanagawa (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/472,476

(22) Filed: May 27, 2009

(30) Foreign Application Priority Data
May 28, 2008 (JP) P2008-138996

(51) Int. Cl. A61B 5/1455

(52) U.S. Cl. 600/322

(57) ABSTRACT

An information processing apparatus includes: an internal information acquisition unit for acquiring pieces of information acquired by a measuring tool attached to a subject's head and indicating blood component changes at measurement portions inside the subject's head; a noise removed signal extracting unit for removing noise components of a brain signal on the basis of a reference signal generated on the basis of information indicating blood component changes at a predetermined measurement portion among the acquired pieces of information indicating blood component changes at the measurement portions, the brain signal being generated on the basis of information indicating blood component changes at the measurement portion different from the predetermined measurement portion; and an output unit for outputting the brain signal, from which the noise components are removed, as a signal indicating an activated state at the corresponding measurement portion of a brain surface layer of the subject.

### BACKGROUND OF THE INVENTION

1. Field of the Invention

The invention relates to an information processing apparatus, information processing method, information processing program, and recording medium and, more particularly, to an information processing apparatus, information processing method, information processing program, and recording medium that are able to accurately measure the activated state of a brain by removing noise components.

2. Description of the Related Art

In recent years, fNIRS (functional near infrared spectroscopy) is employed as a system that measures metabolism of brain blood flow using near infrared light.

For example, a brain activation signal may be measured at a selected target portion of a brain surface layer using fNIRS, and applications to various fields, such as an evaluation technique like various types of emotion estimations or brain function measurements from measurement results or a brain computer interface (hereinafter, referred to as BCI) using motion estimation, have been researched.

In fNIRS, for example, a light-transmitting probe and a light-receiving probe are worn on the head of a subject, near infrared light is irradiated from the light-transmitting probe and is reflected inside the brain of the subject, and then portion of the reflected light is received by the light-receiving probe. Then, the intensity, or the like, of received light is analyzed as a signal to make it possible to measure changes in blood flow, or the like, inside the brain. In fNIRS, such measurement using the light-transmitting probe and the light-receiving probe is performed at various portions of the head of the subject. In addition, such measurement using the light-transmitting probe and the light-receiving probe is performed while changing the wavelength of near infrared light.

Through the above described measurement using fNIRS, for example, changes in the amount of hemoglobin in blood, or the like, are measured at local portions inside the brain, and the activation states of the local portions may be estimated on the basis of the measurement results.

However, a measured signal indicating changes in the amount of hemoglobin inside the brain may include various types of superimposed noise, such as device noise and a body motion signal, from the outside in addition to a signal from the inside of the brain.

Then, it has been suggested that principal component analysis or independent component analysis is carried out on a signal indicating changes in the amount of blood inside the brain, a statistically uncorrelated signal or a signal independent in terms of probability density is extracted, and then the extracted result is displayed.

Patent Document 1: Japanese Unexamined Patent Application Publication No. 2005-143609

### SUMMARY OF THE INVENTION

However, signals indicating changes in the amount of hemoglobin, or the like, in the brain, measured by fNIRS, presumably not only include the influence of oxygen metabolism inside capillary vessels of the brain but also mixedly include the influence of changes in blood flow inside vessels of the scalp. That is, it has proven through measurement experiment that, when noise from the outside is removed, noise that occurs depending on a situation inside the body of the subject is still superimposed on the measurement results of fNIRS.

For this reason, to further accurately carry out measurement using fNIRS, it may be necessary to remove noise components due to the influence of blood flow in the scalp, or the like, from measured signals.

It is desirable to accurately measure the activated state of the brain by removing noise components.

According to an embodiment of the invention, an information processing apparatus includes: internal information acquisition means for acquiring pieces of information that are acquired by a measuring tool attached to a head of a subject and that indicate changes in blood components at measurement portions inside the head of the subject; noise removed signal extracting means for removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and output means for outputting the brain signal, from which the noise components are removed, as a signal indicating an activated state at the corresponding measurement portion of a brain surface layer of the subject.

The information processing apparatus may further include coefficient determination means for determining, on the basis of the reference signal and the brain signal that are acquired in a state where no specific stimulus is applied to the subject, coefficients of a relational expression for obtaining an output value of the brain signal at each measurement portion on the basis of an output value of the reference signal, wherein the noise removed signal extracting means may obtain noise components included in the brain signal on the basis of the determined coefficients to remove the noise components.

The measuring tool may be formed of a light-emitting probe that emits near infrared light and a light-receiving probe that receives near infrared light emitted from the light-emitting probe, wherein the light-emitting probe and the light-receiving probe may be attached at a position corresponding to each measurement portion of the head of the subject, and wherein a distance between the light-emitting probe and the light-receiving probe that are used to generate the reference signal may be shorter than a distance between the light-emitting probe and the light-receiving probe for acquiring information used to generate the brain signal.

Measurement portions for a plurality of the reference signals may be set in correspondence with the respective measurement portions of the brain signals.

Among the measurement portions of the brain signals, distances between the light-emitting probes and the light-receiving probes at predetermined measurement portions may be made short, whereby the predetermined measurement portions may serve as the measurement portions for the reference signals.

One measurement portion for the reference signal may be set at a predetermined position.

The measurement portion for the reference signal may be set at a position corresponding to a longitudinal fissure of cerebrum of the brain in the head of the subject.

The measurement portion for the reference signal may be set at a position 20 mm to 30 mm above a glabella of the subject and at a forehead of the subject with no hair.

According to another embodiment of the invention, an information processing method includes the steps of: acquiring pieces of information that are acquired by a measuring tool attached to a head of a subject and that indicate changes in blood components at measurement portions inside the head of the subject; removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and outputting the brain signal, from which the noise components are removed, as a signal indicating an activated state at the corresponding measurement portion of a brain surface layer of the subject.

According to further another embodiment of the invention, a program is provided for causing a computer to function as an information processing apparatus including: internal information acquisition means for acquiring pieces of information that are acquired by a measuring tool attached to a head of a subject and that indicate changes in blood components at measurement portions inside the head of the subject; noise removed signal extracting means for removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and output means for outputting the brain signal, from which the noise components are removed, as a signal indicating an activated state at the corresponding measurement portion of a brain surface layer of the subject.

According to yet another embodiment of the invention, pieces of information that are acquired by a measuring tool attached to a head of a subject and that indicate changes in blood components at measurement portions inside the head of the subject are acquired; noise components of a brain signal on the basis of a reference signal generated on the basis of information indicating changes in blood components at a predetermined measurement portion among the acquired pieces of information indicating changes in blood components at the measurement portions are removed, wherein the brain signal is generated on the basis of information indicating changes in blood components at the measurement portion different from the predetermined measurement portion; and the brain signal, from which the noise components are removed, is output as a signal indicating an activated state at the corresponding measurement portion of a brain surface layer of the subject.

### BRIEF DESCRIPTION OF THE DRAWINGS

FIG. 1 is a block diagram that shows an example of the configuration of an information processing system according to an embodiment of the invention;

FIG. 2 is a block diagram that shows an example of the configuration of a measuring device shown in FIG. 1;

FIG. 3 is a view for illustrating that signals indicating changes in the amount of hemoglobin, or the like, in a brain, measured using fNIRS, mixedly include the influence of changes in the amount of blood flow inside vessels of a scalp;

FIG. 4 is a block diagram that shows an example of the functional configuration of a control unit shown in FIG. 2;

FIG. 5 is a view that illustrates positions at which light-emitting probes and light-receiving probes are attached when brain signals are measured by the measuring device;

FIG. 6 is a view that shows an example of positions at which light-emitting probes and light-receiving probes are attached when brain signals are measured according to an existing method;

FIG. 7 is a view that shows an example of positions at which light-emitting probes and light-receiving probes are attached when brain signals and a reference signal are measured according to the embodiment of the invention;

FIG. 8 is a view for illustrating positions at which light-emitting probes and light-receiving probes are attached when brain signals and a reference signal of the frontal lobe of a subject are measured;

FIG. 9 is a graph that represents the relationship between an output value of the reference signal and an output value of the brain signal, which are samples at a measurement channel;

FIG. 10 is a view that shows an example of brain signals and a reference signal that are acquired in a state where no specific stimulus is applied to the subject;

FIG. 11 is a view that shows an example of the case where noise of brain signals at the measurement channels shown in FIG. 10 is removed;

FIG. 12 is a view that shows an example of brain signals and a reference signal that are acquired in a state where a specific stimulus is applied to the subject;

FIG. 13 is a view that shows an example of the case where noise of brain signals at the measurement channels shown in FIG. 12 is removed;

FIG. 14 is a flowchart that illustrates an example of a pre-measurement process;

FIG. 15 is a flowchart that illustrates an example of a sample acquisition process;

FIG. 16 is a flowchart that shows a coefficient determination process;

FIG. 17 is a flowchart that illustrates a brain signal extracting process;

FIG. 18 is a view that shows another example of positions at which light-emitting probes and light-receiving probes are attached when brain signals and reference signals are measured according to the embodiment of the invention;

FIG. 19 is a view that shows further another example of positions at which light-emitting probes and light-receiving probes are attached when brain signals and reference signals are measured according to the embodiment of the invention;

FIG. 20 is a table that illustrates advantageous effects according to the embodiment of the invention;

FIG. 21 is a graph that corresponds to the table of FIG. 20; and

FIG. 22 is a block diagram that shows an example of the configuration of a personal computer.

### DESCRIPTION OF THE PREFERRED EMBODIMENTS

Hereinafter, an embodiment of the invention will be described with reference to the accompanying drawings.

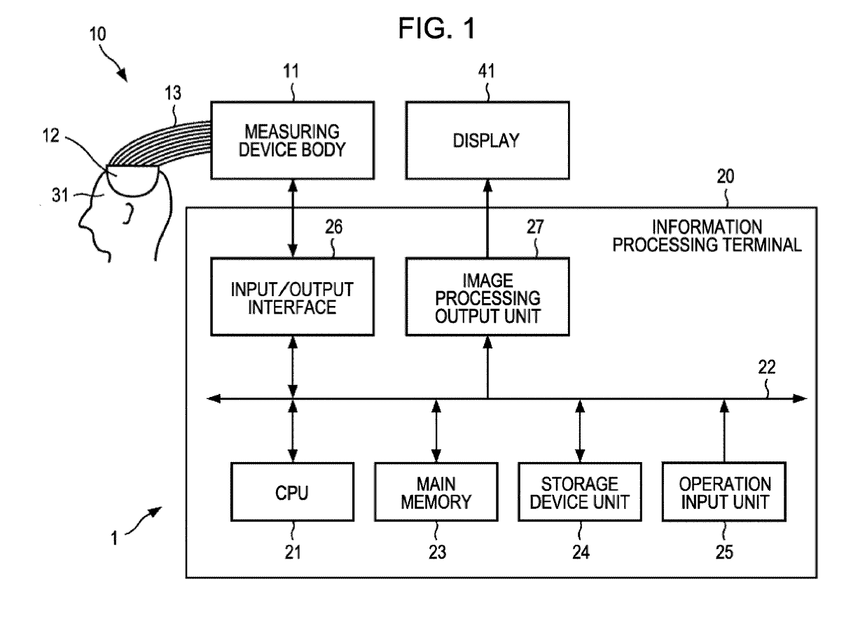

FIG. 1 is a block diagram that shows an example of the configuration of an information processing system according to an embodiment of the invention.

In this example, the information processing system 1 includes a measuring device 10, an information processing terminal 20 and a display 41.

The measuring device 10 measures hemoglobin concentration or changes in hemoglobin concentration in blood flow in a brain surface layer of a subject 31 by means of functional near infrared spectroscopy, and is, for example, formed of an fNIRS (functional near infrared spectroscopy). Although it will be described later in detail, the measuring device 10 includes a measuring device body 11, a measuring tool 12 worn on the head of the subject 31, and a cable 13 that connects the measuring device body 11 with the measuring tool 12.

The information processing terminal 20 is formed of a personal computer, a mobile computer, or the like.

The information processing terminal 20 includes a CPU 21. The information processing terminal 20 includes a bus 22 to which a main memory 23, a storage device unit 24, and an operation input unit 25 are connected. Programs or data are deployed in the main memory 23. The storage device unit 24 is formed of a nonvolatile storage medium, such as a hard disk.

A program for executing process of estimating the brain activation state, or the like, according to the embodiment of the invention, necessary fixed data, and the like, are recorded in the storage device unit 24.

In addition, an input/output interface 26 and an image processing output unit 27 are connected to the bus 22, the measuring device body 11 is connected to the input/output interface 26, and the display 41 is connected to the image processing output unit 27.

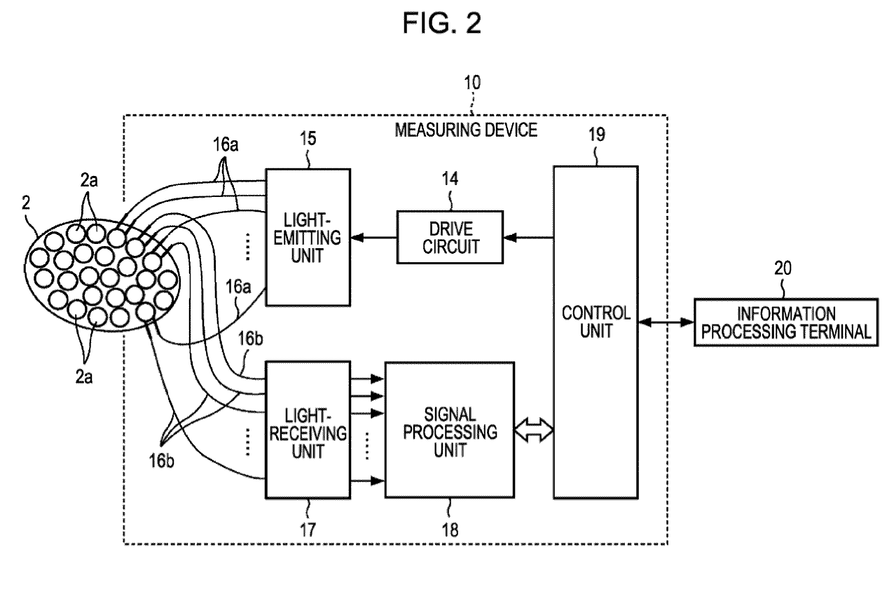

FIG. 2 is a block diagram that shows an example of the configuration of the measuring device 10 shown in FIG. 1.

In the measuring device 10, a plurality of portions of the brain surface layer 2 of the subject 31 are respectively set as measurement portions 2a. In order to show information, indicating which portion (measurement portion) of the brain surface layer 2 of the subject 31 is activated to what degree, to the user of the information processing system 1, the measuring device 10 measures hemoglobin concentration, or the like, in blood that flows inside vessels at the measurement portions of the brain surface layer 2 of the subject 31.

In the measuring device 10, near infrared light emitted from a light-emitting unit 15, which is driven by a drive circuit 14, is irradiated to the respective measurement portions 2a through optical fibers 16a that constitute light-emitting probes. Note that each of the light-emitting probes is actually attached so as to be in close contact with the head (scalp) of the subject 31.

Near infrared light irradiated to each measurement portion 2a transmits through each measurement portion 2a and exits outside the head of the subject 31 depending on hemoglobin concentration or changes in hemoglobin concentration in blood flow at each measurement portion 2a.

Each ray of light exiting outside the head of the subject 31 is received by a light-receiving unit 17 through a corresponding one of optical fibers 16b that constitute light-receiving probes, and converted into a light-receiving signal, which is an electric signal. Thus, each light-receiving probe receives light of an intensity corresponding to hemoglobin concentration in blood flow at the corresponding measurement portion 2a, and an electric signal corresponding to the intensity of light received is generated.

Each light-receiving signal is converted into a measured value in digital data by the signal processing unit 18, and is filtered to remove noise components coming from the outside of the brain of the subject 31, such as device noise and body motion, and is then transmitted to the information processing terminal 20 through a control unit 19.

The control unit 19 controls various types of processes executed by the measuring device 10.

The measurement portions 2a are set in correspondence with measurement channels of signals measured by the measuring device 10. For example, a light-receiving signal measured at the first measurement portion 2a serves as a signal of a first measurement channel, a light-receiving signal measured at the second measurement portion 2a serves as a signal of a second measurement channel, and the like. Signals at the respective local measurement portions (measurement portions) in the brain surface layer 2 of the subject serve as signals of respective measurement channels.

By analyzing light-receiving signals of the light-receiving unit 17, changes in oxygenated hemoglobin concentration-related value, changes in deoxygenated hemoglobin concentration-related value, and the like, are measured at the respective measurement portions 2a. Alternatively, by analyzing light-receiving signals of the light-receiving unit 17, it is possible to measure blood flow (changes in the amount of blood) at the respective measurement portions 2a.

In this way, by analyzing acquired signals of the respective measurement channels, it is possible to estimate emotion, or the like, of the subject.

However, signals indicating changes in the amount of hemoglobin, or the like, in the brain, measured by fNIRS, presumably not only include the influence of oxygen metabolism inside capillary vessels of the brain but also mixedly include the influence of changes in blood flow inside vessels of the scalp. That is, it has proven through measurement experiment that noise that occurs depending on a situation inside the body of the subject is superimposed on the measurement results of fNIRS.

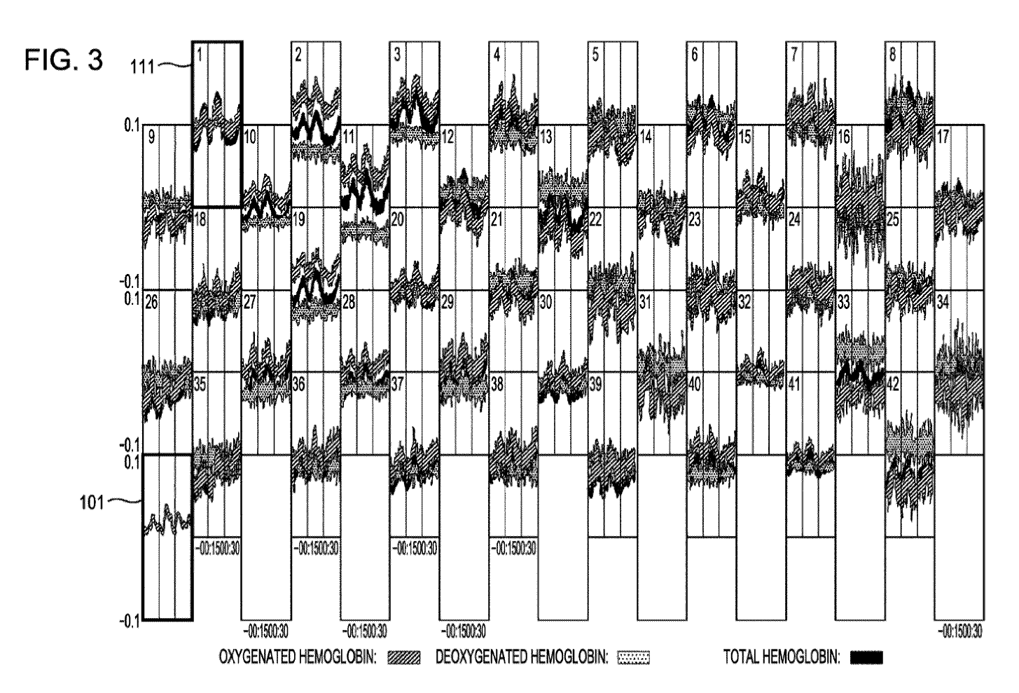

FIG. 3 is a view for illustrating that signals indicating changes in the amount of hemoglobin, or the like, in the brain, measured using fNIRS, mixedly include the influence of changes in the amount of blood flow inside vessels of the scalp. In the drawing, a plurality of graphs are shown. A graph 101 at the lower left in the drawing shows changes in blood flow in the scalp of the subject, measured by a laser Doppler blood-flowmetry. Graphs other than the graph 101 in FIG. 3 show signals of respective measurement channels and indicate changes in the amount of oxygenated hemoglobin, changes in the amount of deoxygenated hemoglobin and changes in the total amount of hemoglobin at measurement portions corresponding to the respective measurement channels. Numbers assigned to the graphs of the signals of the respective measurement channels represent the measurement channel numbers. In this example, graphs of first to forty-second measurement channels are shown.

Note that in each of the graphs in FIG. 3, the abscissa axis represents time and the ordinate axis represents a variation in amount, and it is assumed that signals of the respective measurement channels are measured at the same time as the time of measurement of the graph 101.

For example, when the graph 101 that represents changes in blood flow in the scalp of the subject is compared with a graph 111 of the first measurement channel, it appears that waveforms resemble each other. That is, the graph 101 and the graph 111 are highly correlative.

Note that it is described in FIG. 3 that the graph 101 shows changes in blood flow of the scalp of the subject, measured by a laser Doppler blood-flowmetry; instead, for example, changes in the amount of hemoglobin, as well as signals of the measurement channels, may be measured. In short, it describes that changes in blood components inside vessels of the scalp portion that does not reach a brain epidermis of the subject greatly influences changes in blood components inside vessels of the brain epidermis or brain of the subject.

In addition, as well as the graph 111, many waveforms of other graphs also resemble the waveform of the graph 101. For this reason, it is conceivable that there is a high correlation among changes in blood flow of the scalp, changes in the amount of oxygenated hemoglobin, and changes in the total amount of hemoglobin.

Thus, for example, even when a waveform that represents changes in the amount of hemoglobin at a predetermined channel indicates steep vertical variations, but when the blood flow in the scalp largely changes at the same timing, if emotion of the subject is estimated on the basis of the measurement result of hemoglobin at that predetermined channel, it is highly likely to obtain an erroneous result.

Then, in the embodiment of the invention, changes in the amount of blood that flows in vessels outside the brain, such as vessels in the scalp, are recognized as noise, and the noise components are removed to make it possible to obtain accurate measurement results.

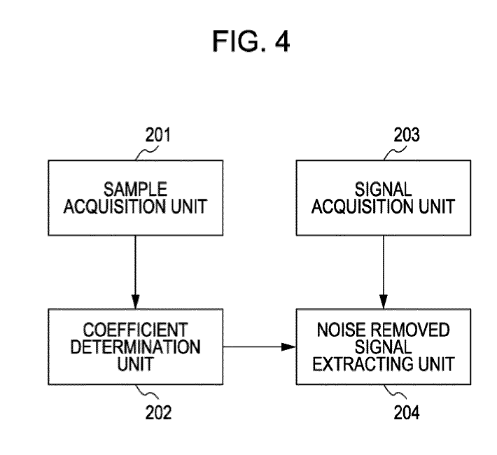

FIG. 4 is a block diagram that shows an example of the functional configuration of the control unit 19 shown in FIG. 2. A sample acquisition unit 201 and a coefficient determination unit 202 in the drawing are functional blocks that, for example, execute preprocessing prior to actual measurement of signals for estimating emotion of the subject.

The sample acquisition unit 201, for example, controls the drive circuit 14 at a timing instructed by the user to cause the light-emitting unit 15 to emit near infrared light, and controls the signal processing unit 18 to acquire signals of the respective measurement channels.

At this time, the light-emitting probes and the light-receiving probes are attached so that at least one of the measurement channels measures changes in the amount of hemoglobin in vessels of the scalp that does not reach the brain epidermis of the subject. In this way, an acquired signal that represents changes in the amount of hemoglobin in vessels of the scalp outside the brain epidermis of the subject is referred to as a reference signal. On the other hand, the other acquired signals of the measurement channels, which represent changes in the amount of hemoglobin in vessels of the brain epidermis or brain of the subject, are referred to as brain signals. Although it will be described later in detail, the value of the reference signal does not allow direct measurement of the activated state at each measurement portion of the brain surface layer of the subject but the value is referred to in order to obtain noise components of the brain signals at respective measurement portions of the brain surface layer.

The sample acquisition unit 201 stores the thus acquired reference signal and brain signals. The sample acquisition unit 201 stores a plurality of samples, which, for example, have combinations of a reference signal and brain signals. The combinations are respectively acquired at different timings.

The coefficient determination unit 202, for example, generates primary expressions, by which brain signal values are obtained from a reference signal value, on the basis of the sample acquired by the sample acquisition unit 201, and determines the coefficients of the primary expression using least square method, or the like. Note that the detail of the process executed by the coefficient determination unit 202 will be described later.

A signal acquisition unit 203 and a noise removed signal extracting unit 204 shown in FIG. 4 are functional blocks that, for example, execute process of actually measuring signals for estimating emotion, or the like, of the subject.

The signal acquisition unit 203, as well as the sample acquisition unit 201, for example, controls the drive circuit 14 at a timing instructed by the user to cause the light-emitting unit 15 to emit near infrared light, and controls the signal processing unit 18 to acquire signals of the respective measurement channels. Then, the signal acquisition unit 203 acquires a reference signal and brain signals simultaneously.

Note that the signal acquisition unit 203 and the sample acquisition unit 201 may be formed as one unit.

The noise removed signal extracting unit 204 processes removal of noise components from the brain signals acquired by the signal acquisition unit 203 on the basis of the reference signal acquired by the signal acquisition unit 203 and the coefficients determined by the coefficient determination unit 202. Then, the noise removed signal extracting unit 204, for example, outputs the brain signals, from which noise components are removed, as signals for estimating emotion, or the like, of the subject.

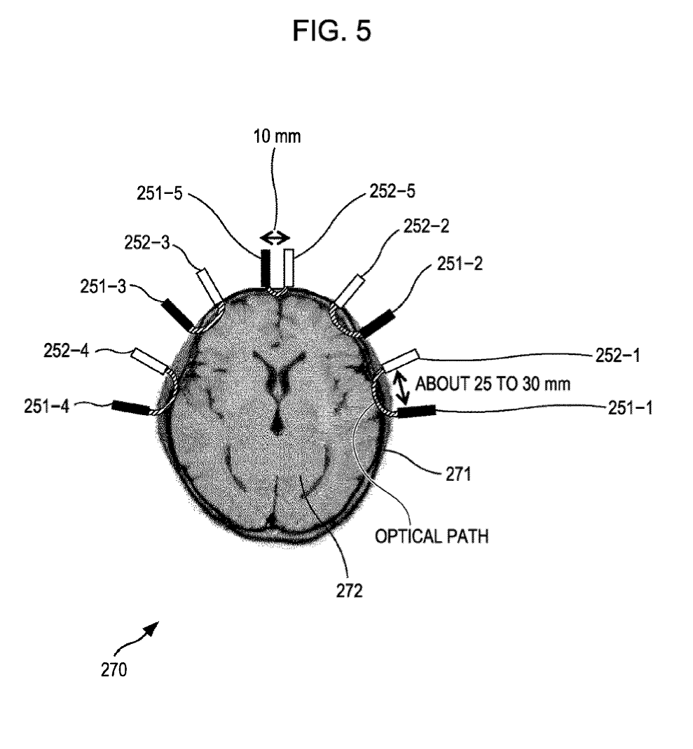

FIG. 5 is a view that illustrates positions at which light-emitting probes and light-receiving probes are attached when brain signals are measured by the measuring device 10. In the drawing, a cross-sectional view is shown when a head 20 of the subject is viewed from the upper side.

Black rectangles shown in the drawing represent light-emitting probes, and white rectangles represent light-receiving probes. Note that, as described with reference to FIG. 2, actually, the light-emitting probes and the light-receiving probes are connected to the measuring device body 11 by optical fibers; however, the optical fibers are not shown in FIG. 5.

A pair of light-emitting probe 251-1 and light-receiving probe 252-1 are attached to the scalp 271 of the subject. Near infrared light emitted from the light-emitting probe 251-1 transmits through the scalp 271, the cranium, and the like, to the brain 272 of the subject, and portion of the near infrared light that reaches the brain 272 travels along the optical path indicated by hatching in the drawing and is received by the light-receiving probe 252-1. Then, light received by the light-receiving probe 252-1 is received by the light-receiving unit 17 via the optical fiber 16b as described above, and converted into a light-receiving signal, which is an electric signal. A brain signal is generated on the basis of the light-receiving signal. For example, the pair of light-emitting probe 251-1 and light-receiving probe 251-2 measure a brain signal of a first measurement channel.

Similarly, pairs of light-emitting probe 251-2 to light-emitting probe 251-4 and light-receiving probe 252-2 to light-receiving probe 252-4 are attached to the scalp 271 of the subject. Then, as in the case of the above, a brain signal is generated on the basis of a light-receiving signal. For example, the pairs of light-emitting probe 251-2 to light-emitting probe 251-4 and light-receiving probe 252-2 to light-receiving probe 252-4 measure brain signals of the second measurement channel to the fourth measurement channel.

In FIG. 5, the optical paths of the respective pairs of light-emitting probe 251-2 to light-emitting probe 251-4 and light-receiving probe 252-2 to light-receiving probe 252-4 reach the brain 272; however, positions of the optical paths differ depending on a distance between the light-emitting probe and the light-receiving probe. Specifically, depending on a distance between the light-emitting probe and the light-receiving probe, the optical path reaches a deep portion toward the center of the head 270 of the subject or the optical path reaches just a portion near the surface of the head 270.

For example, as the distance between the light-emitting probe and the light-receiving probe increases, the optical path reaches a deeper portion inside the brain 272. On the other hand, as the distance between the light-emitting probe and the light-receiving probe is reduced, the optical path just reaches the surface layer of the brain 272. That is, by adjusting the distance between the light-emitting probe and the light-receiving probe, for example, the measurement portion may be located at the surface layer of the brain 272 or the measurement portion may be located at a portion that does not reach the surface layer of the brain 272 but inside the scalp 271.

In the embodiment of the invention, to acquire the above described reference signal, the light-emitting probe 251-5 and the light-receiving probe 252-5 are attached to the scalp 271 of the subject. The distance between the light-emitting probe 251-5 and the light-receiving probe 252-5 is shorter than the distances between the light-emitting probe 251-1 to the light-emitting probe 251-4 and the light-receiving probe 252-1 to the light-receiving probe 252-4.

Near infrared light emitted from the light-emitting probe 251-5 reaches the inside of the scalp 271, portion of the near infrared light travels along the optical path indicated by hatching in the drawing, and is then received by the light-receiving probe 252-5. That is, the optical path of the pair of light-emitting probe 251-5 and light-receiving probe 252-5 does not reach the epidermis of the brain 272. Thus, it is possible to generate the above described reference signal on the basis of the light-receiving signal of light received by the light-receiving probe 252-5.

It is desirable that the distances between the light-emitting probe 251-1 to the light-emitting probe 251-4 and the light-receiving probe 252-1 to the light-receiving probe 252-4 are, for example, 25 mm to 30 mm. In addition, it is desirable that the distance between the light-emitting probe 251-5 and the light-receiving probe 252-5 is, for example, about 10 mm.

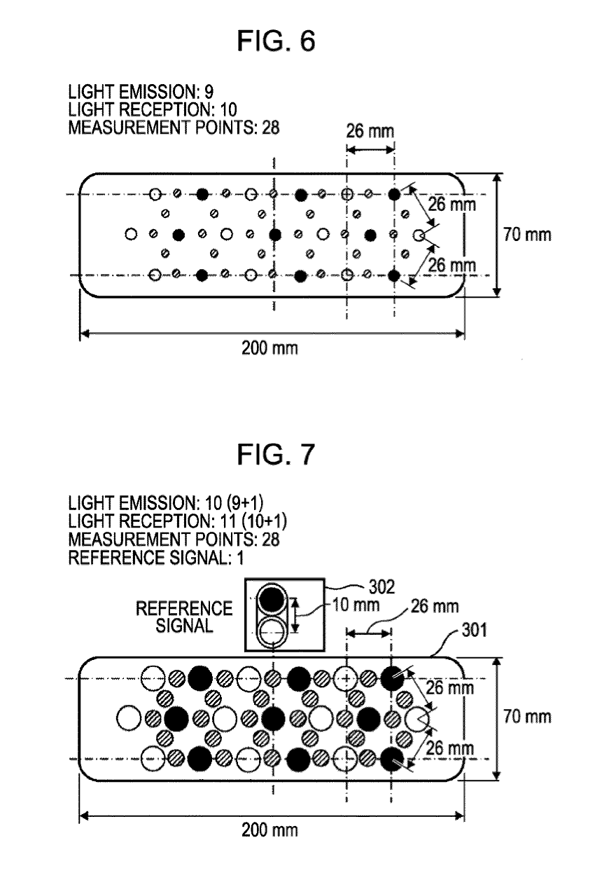

FIG. 6 is a view that shows an example of positions at which light-emitting probes and light-receiving probes are attached when brain signals are measured according to an existing method. Black circles in the drawing represent positions at which the light-emitting probes are attached, white circles in the drawing represent positions at which the light-receiving probes are attached, and hatched circles represent optical paths. In the example of FIG. 6, nine light-emitting probes and ten light-receiving probes are attached in a region of 70 mm in the vertical direction and 200 mm in the horizontal direction in the drawing on the scalp of the subject, and 28 measurement portions are set in total. Thus, near infrared light emitted from one light-emitting probe may possibly be received by two light-receiving probes; however, a manner in which brain signals are generated is not different from the above manner described with reference to FIG. 5.

In this example, the distances between the light-emitting probes and the light-receiving probes each are set at 26 mm.

FIG. 7 is a view that shows an example of positions at which light-emitting probes and light-receiving probes are attached when brain signals and a reference signal are measured according to the embodiment of the invention. Black circles in the drawing represent positions at which the light-emitting probes are attached, white circles in the drawing represent positions at which the light-receiving probes are attached, and hatched circles represent optical paths. Note that the positions of the optical paths correspond to measurement portions in the brain surface layer of the subject. In the example of the drawing, as in the case of FIG. 6, nine light-emitting probes and ten light-receiving probes are attached in a region of 70 mm in the vertical direction and 200 mm in the horizontal direction in the drawing on the scalp of the subject, and 28 measurement portions are set in total.

Note that the size of the region 301 in the drawing is an example, and the vertical length and/or horizontal length of the region 301 in the drawing vary depending on the number of measurement portions, or the like.

In addition, in the example of FIG. 7, one light-emitting probe and one light-receiving probe are attached to a region 302 on the upper side in the drawing of the region 301, and the pair of light-emitting probe and light-receiving probe are used to generate a reference signal. Although not shown in the drawing, of course, an optical path exists between the light-emitting probe and the light-receiving receiving probe for generating a reference signal.

In this example, the distances between the light-emitting probes and the light-receiving probes that are attached inside the region 301 each are set at 26 mm. That is, the distances between the light-emitting probes and the light-receiving probes, which are used to generate brain signals, each are set at 26 mm. On the other hand, the distance between the light-emitting probe and the light-receiving probe, which are used to generate a reference signal, is set at 10 mm.

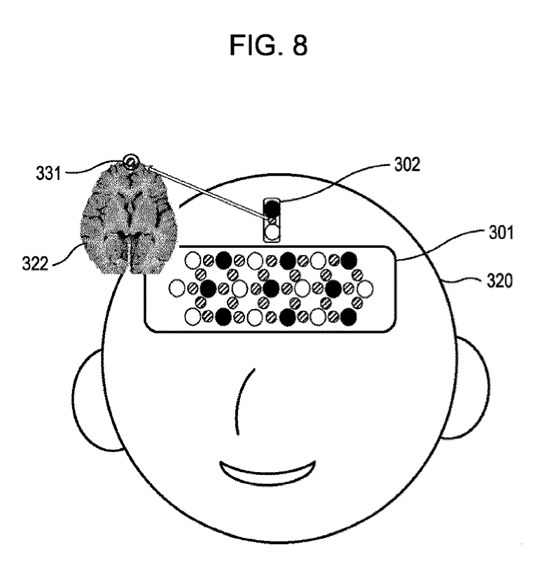

FIG. 8 is a view for illustrating positions at which light-emitting probes and light-receiving probes are attached when brain signals and reference signals of the frontal lobe of the subject are measured. FIG. 8 is a view when the head 320 of the subject is viewed from the front (face), and the region 301 is set at a position corresponding to the frontal lobe of the subject, that is, the forehead. The light-emitting probes and the light-receiving probes attached to the region 301 are similar to the case described with reference to FIG. 7. In addition, the region 302 is set on the upper side of the region 301, and the light-emitting probe and the light-receiving probe are attached to the region 302 so that they are arranged vertically in the drawing.

It is desirable that the region 302 is set at the longitudinal fissure of cerebrum of the brain of the subject. The drawing at the upper left in FIG. 8 is a view for illustrating the position of the region 302. A vertical groove is formed in the brain 322 of the subject when a line that connects both eyes of the subject is horizontal, and the groove is called longitudinal fissure of cerebrum. Because the longitudinal fissure of cerebrum is a groove, the epidermis of the brain 322 is recessed toward the center of the head 320 of the subject at the longitudinal fissure of cerebrum. Thus, because the distance from the scalp of the subject to the epidermis of the brain 322 increases at the longitudinal fissure of cerebrum, it is easy to acquire a reference signal.

Furthermore, it is desirable that the region 302 is set at a position 20 mm to 30 mm upper from the glabella of the subject 31 and at the forehead of the subject with no hair. The region 302 is set at that position because the position 20 mm to 30 mm upper from the glabella of the subject 31 is less likely that the scalp of the forehead largely moves due to changes in facial expression of the subject 31, or the like, and the reference signal may be further accurately acquired. This is also because, if the hair of the subject 31 contacts the light-emitting probe or the light-receiving probe, it is highly likely that an output value of the reference signal cannot be accurately measured.

Next, the detail of the process executed by the coefficient determination unit 202 will be described. As described above, the coefficient determination unit 202, for example, generates primary expressions, by which brain signal values are obtained from a reference signal value, on the basis of the sample acquired by the sample acquisition unit 201, and determines the coefficients of the primary expression using least square method, or the like.

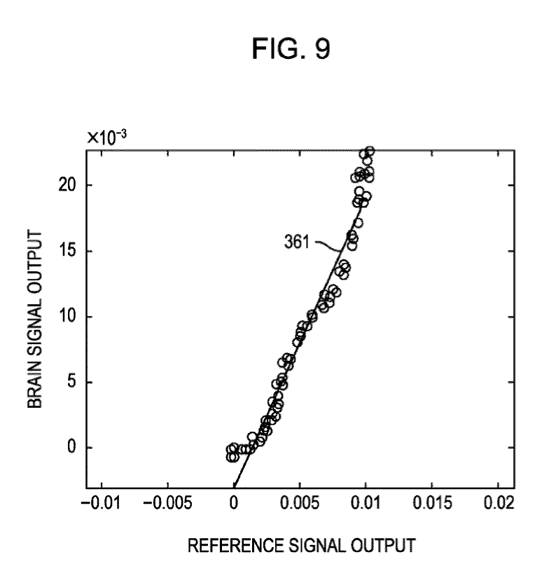

FIG. 9 is a graph that represents the relationship between an output value of the reference signal and an output value of the brain signal, which are samples at a measurement channel. In the drawing, the abscissa axis represents an output value of the reference signal, and the ordinate axis represents an output value of the brain signal. Then, the output values of the brain signals corresponding to the output values of the reference signals, acquired as samples at that measurement channel, are plotted by circle in the drawing.

The coefficient determination unit 202 generates an approximate line on the basis of plotted points shown in FIG. 9. More specifically, the coefficients of primary expression that satisfies the relationship between the reference signal and the brain signal of the n-th measurement channel is obtained.

Now, the reference signal is expressed by Ref(t) as a function of time t, and the brain signal is expressed by Nn(t) as a function of time t. The coefficient determination unit 202 substitutes an output value of the reference signal and an output value of the brain signal at each instant of time into the following mathematical expression.

Nn(t)=An·Ref(t)+Bn

Then, the coefficient determination unit 202 calculates An and Bn using, for example, least square method. Thus, coefficients An and Bn of the n-th measurement channel are determined.

Similarly, the coefficient determination unit 202 executes the above processes using samples of the measurement channels, other than the n-th measurement channel, to determine coefficients of the respective measurement channels. For example, when there are first to 28th measurement channels, A1 to A28 and B1 to B28 are determined.

Note that in this example, as shown in FIG. 9, the approximate line 361 may be generated, so the obtained relational expression is a primary expression; instead, polynomial may be employed as a relational expression depending on the state of distribution of output values of the brain signals and output values of the reference signals. For example, when output values of the brain signals corresponding to output values of reference signals and acquired as samples of a measurement channel are plotted, if those plotted points are not arranged in line, an appropriate polynomial may be employed as the relational expression.

The noise removed signal extracting unit 204 processes removal of noise components from the brain signals acquired by the signal acquisition unit 203 on the basis of the thus determined coefficients of the respective measurement channels.

For example, when the brain signal of the n-th measurement channel, acquired by the signal acquisition unit 203, is expressed by Mn(t) as a function of time t, the noise removed signal extracting unit 204 obtains the noise-removed brain signal Sn(t) of the n-th measurement channel from the following mathematical expression.

Sn(t)=Mn(t)−An·Ref(t)−Bn

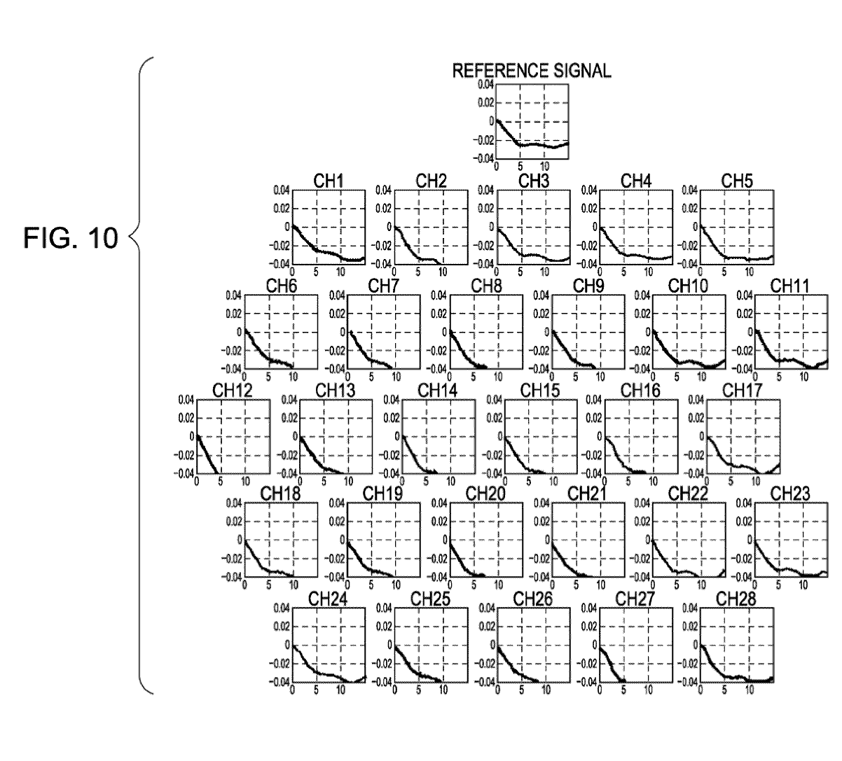

FIG. 10 is a view that shows an example of brain signals and a reference signal that are acquired, for example, in a state where no specific stimulus is applied to the subject. In the drawing, 29 graphs are shown, and, in each graph, the abscissa axis represents time, and the ordinate axis represents an output value of a signal. The graphs indicated by “CH1” to “CH28” are graphs of brain signals of the first measurement channel to the 28th measurement channel, and the graph shown on the uppermost side in the drawing is a graph of a reference signal.

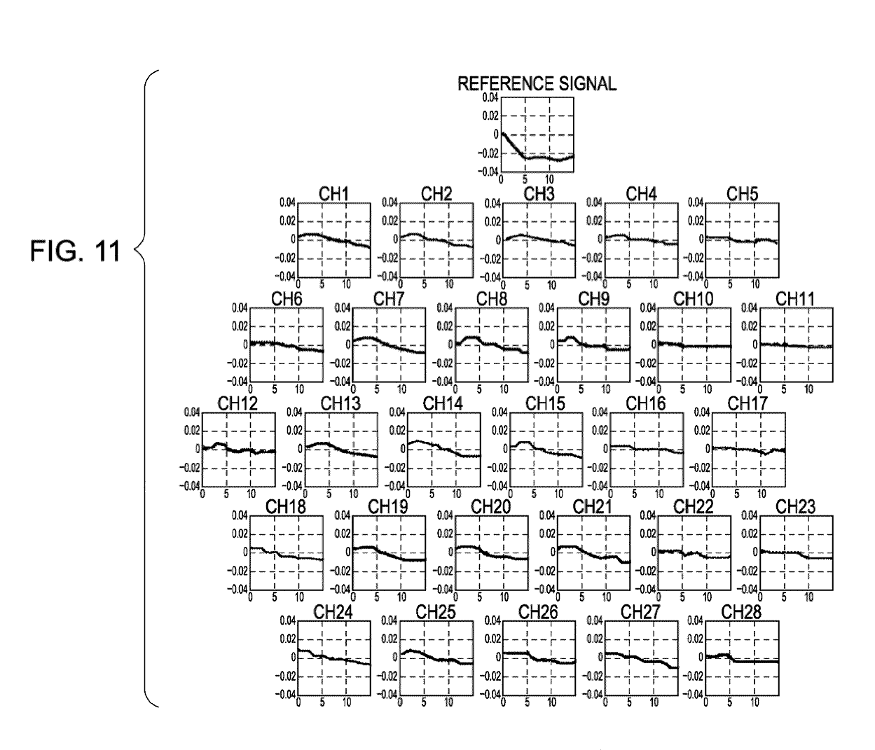

FIG. 11 is a view that shows an example of the case where noise of brain signals of the first measurement channel to the 28th measurement channel shown in FIG. 10 is removed. The graphs of the brain signals of the first measurement channel to the 28th measurement channel shown in FIG. 11 show relatively flat waveforms as compared with the graphs of the brain signals of the first measurement channel to the 28th measurement channel shown in FIG. 10.

The graphs shown in FIG. 10 show the waveforms of the brain signals of the respective measurement channels, acquired in a state where no specific stimulus is applied to the subject. Thus, intrinsically, large changes in the amount of hemoglobin in vessels of the brain should not appear. That is, the waveforms of the graphs of the brain signals of the first measurement channel to the 28th measurement channel in FIG. 10 resemble the shape of the waveform of the graph of the reference signal because of the influence of the reference signal.

For example, signals indicating changes, or the like, in the amount of hemoglobin inside the brain, measured by existing fNIRS, mixedly include the influence of changes in the amount of blood flow inside vessels of the scalp. Thus, even when brain signals are acquired in a state where no specific stimulus is applied to the subject, the waveforms of the graphs may fluctuate vertically. For example, in the graphs of the brain signals of the first measurement channel to the 28th measurement channel in FIG. 10, substantially similar downward-sloping waveforms are shown. It is difficult to accurately estimate, for example, emotion of the subject on the basis of such waveforms.

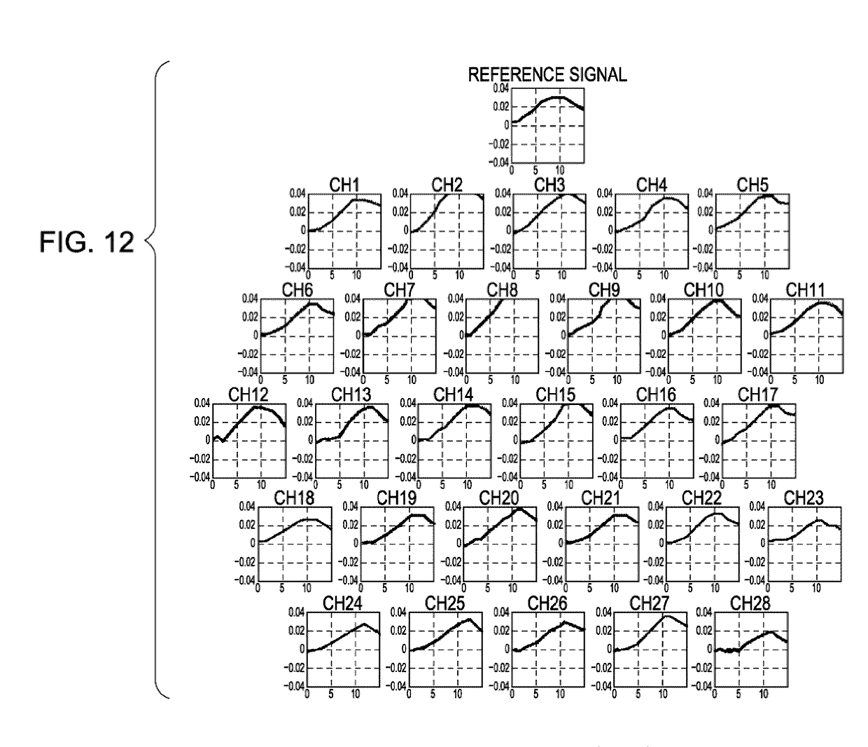

FIG. 12 is a view that shows an example of brain signals and a reference signal that are acquired, for example, in a state where a specific stimulus is applied to the subject. In the drawing, 29 graphs are shown, and, in each graph, the abscissa axis represents time, and the ordinate axis represents an output value of a signal. The graphs indicated by “CH1” to “CH28” are graphs of brain signals of the first measurement channel to the 28th measurement channel, and the graph shown on the uppermost side in the drawing is a graph of a reference signal.

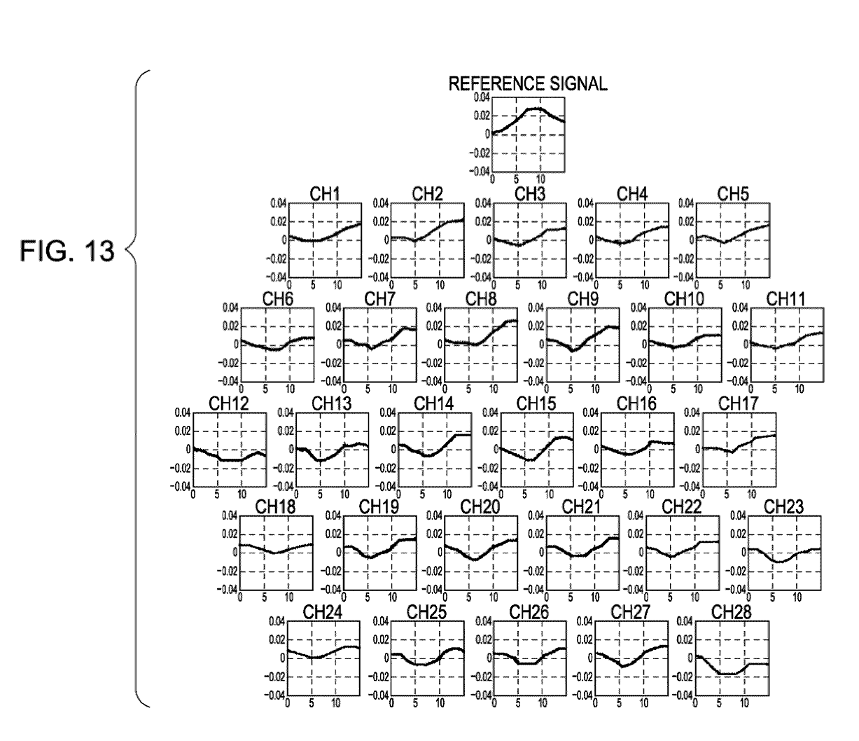

FIG. 13 is a view that shows an example of the case where noise of brain signals of the first measurement channel to the 28th measurement channel shown in FIG. 12 is removed. In the drawing, 29 graphs are shown, and, in each graph, the abscissa axis represents time, and the ordinate axis represents an output value of a signal. The graphs indicated by “CH1” to “CH28” are graphs of brain signals of the first measurement channel to the 28th measurement channel, and the graph shown on the uppermost side in the drawing is a graph of a reference signal.

The waveforms of the graphs of the brain signals of the first measurement channel to the 28th measurement channel in FIG. 12 show the shapes in which substantially similar waveforms that once go upward from the left to right and then go downward. On the other hand, the waveforms of the graphs of the brain signals of the first measurement channel to the 28th measurement channel in FIG. 13 respectively have different shapes.

That is, the waveforms of the graphs of the brain signals of the first measurement channel to the 28th measurement channel in FIG. 12 resemble the shape of the waveform of the graph of the reference signal because of the influence of the reference signal. Then, the influence of the reference signal is recognized as noise, and the noise is removed. Thus, as shown in FIG. 13, it is possible to observe changes in the amount of hemoglobin inside vessels of the brain, corresponding to the stimulus applied to the subject, as the brain signals of the respective measurement channels corresponding to the measurement portions.

In this way, the relational expression (including coefficients) that represents the relationship between output values of the reference signals and output values of the brain signals in a state where no specific stimulus is applied to the subject is obtained beforehand, values obtained from the relational expression are regarded as noise components to remove noise from the measured brain signals. In this way, when emotion of the subject is estimated on the basis of the waveforms from which noise is removed, it is possible to further accurately estimate emotion of the subject.

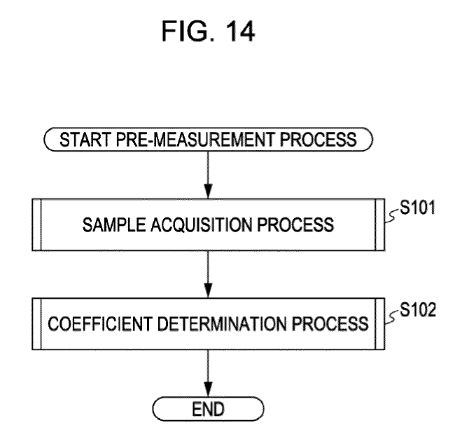

Next, pre-measurement process executed by the information processing system 1 according to the embodiment of the invention will be described with reference to the flowchart of FIG. 14. The process is, for example, executed prior to actual measurement of signals for estimating emotion of the subject.

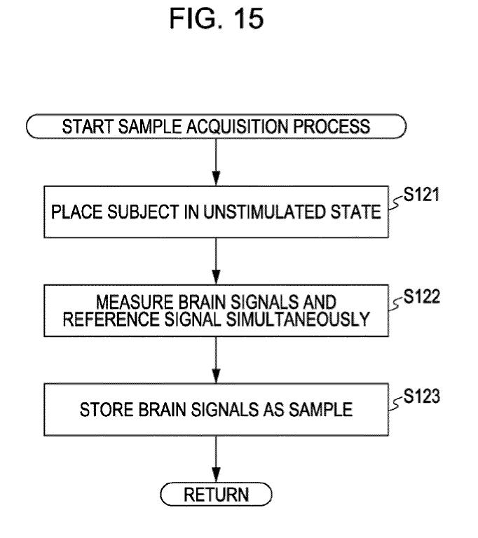

In step S101, the sample acquisition unit 201 executes sample acquisition process, which will be described later with reference to FIG. 15.

Here, the detail of the sample acquisition process in step S101 of FIG. 14 will be described with reference to the flowchart of FIG. 15.

In step S121, the user of the information processing system 1 places the subject in an unstimulated state where no specific stimulus is applied. Then, the user instructs the measuring device 10 to acquire a sample.

In step S122, the sample acquisition unit 201 acquires brain signals and a reference signal simultaneously. At this time, for example, the brain signals of the respective measurement channels corresponding to the measurement portions are acquired together with the reference signal.

In step S123, the sample acquisition unit 201 stores the brain signals of the respective measurement channels, acquired in the process of step S122, in correspondence with the reference signal as a sample.

Note that the number of samples is not limited to one; instead, a plurality of samples may be acquired. That is, the processes of steps S121 to S123 may be, for example, repeatedly executed to acquire a plurality of samples.

In this way, samples are acquired.

Referring back to FIG. 14, after the process of step S101, the process proceeds to step S102.

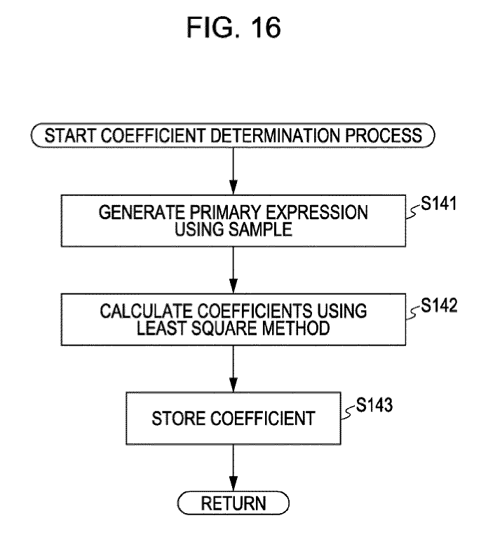

In step S102, coefficient determination process, which will be described with reference to FIG. 16, is executed. Here, the detail of the coefficient determination process in step S102 of FIG. 14 will be described with reference to the flowchart of FIG. 16.

In step S141, the coefficient determination unit 202 generates primary expressions using the sample acquired through the process of step S101. At this time, for example, as described with reference to FIG. 9, specifically, a function that represents the relationship between the reference signal and the brain signal of the n-th measurement channel is generated.

As described above, where the reference signal is expressed by Ref(t) as a function of time t, and the brain signal is expressed by Nn(t) as a function of time t, the coefficient determination unit 202 generates primary expression by substituting an output value of the reference signal and an output value of the brain signal at each instant of time into the following expression.

Nn(t)=An·Ref(t)+Bn

In step S142, the coefficient determination unit 202 solves the primary expression generated in step S141 using, for example, least square method to calculate a coefficient An and a coefficient Bn. Thus, the coefficient An and coefficient Bn of the n-th measurement channel are determined. Similarly, the coefficient determination unit 202 executes the above processes using samples of the measurement channels, other than the n-th measurement channel, to determine coefficients of the respective measurement channels.

In step S143, the coefficient determination unit 202 stores the coefficients of each measurement channel, determined in step S142.

In this way, the coefficient determination process is executed.

In this way, the functions that represent the relationship of the brain signals are generated on the basis of the samples acquired in advance, and then the coefficients of the function are determined. Thus, it is possible to process removal of reference signal components from the brain signals measured later.

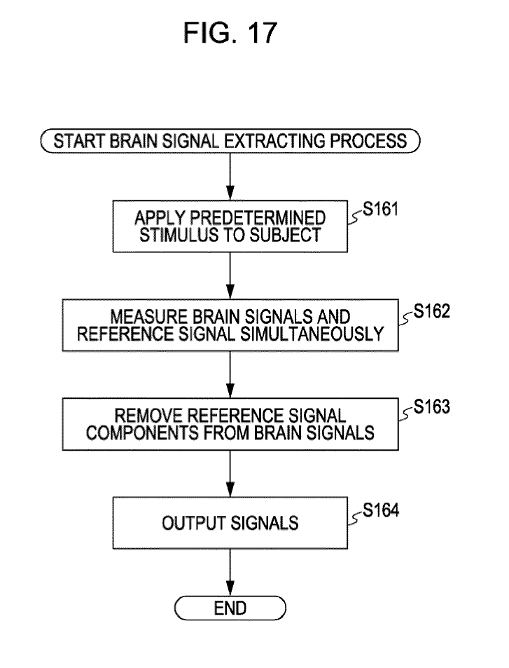

Next, brain signal extracting process executed by the information processing system 1 according to the embodiment of the invention will be described with reference to the flowchart of FIG. 17. This process is, for example, executed to actually measure signals for estimating emotion, or the like, of the subject after the pre-measurement process described with reference to FIG. 14 is executed.

In step S161, the user of the information processing system 1 applies a predetermined stimulus to the subject. At this time, for example, a stimulus for recalling emotion, such as “pleasure” and “sorrow”, is applied to the subject. Then, the user instructs the measuring device 10 to extract brain signals.

In step S162, the signal acquisition unit 203 acquires brain signals and a reference signal simultaneously. At this time, for example, the brain signals of the respective measurement channels corresponding to the measurement portions are acquired together with the reference signal.

In step S163, the noise removed signal extracting unit 204 removes reference signal components (that is, noise components) from the brain signals. At this time, the noise removed signal extracting unit 204, for example, reads the coefficients of each measurement channel, stored in the process of step S143 of FIG. 16, to process removal of noise components from the brain signals acquired in the process of step S162.

For example, if the brain signal of the n-th measurement channel, acquired by the signal acquisition unit 203 in the process of step S162, is defined by Mn(t) as a function of time t, the noise removed signal extracting unit 204 obtains the brain signal Sn(t) of the n-th measurement channel, from which noise is removed, using the following mathematical expression.

Sn(t)=Mn(t)−An·Ref(t)−Bn

In this way, noise in the brain signals of all the measurement channels is removed.

In step S164, the noise removed signal extracting unit 204 outputs the brain signals of the respective measurement channels, from which noise is removed in the process of step S163, as ultimate brain signals. Here, the output signals are used as signals for representing the activated state at each measurement portion of the brain surface layer of the subject.

In this way, the noise-removed brain signals are extracted.

Thus, in the embodiment of the invention, changes in the amount of blood that flows in vessels outside the brain, such as vessels of the scalp, are acquired as a reference signal, and the reference signal is removed from the brain signals as noise components. Thus, for example, without any influence of changes, or the like, in the amount of blood that flows in vessels outside the brain, such as vessels in the scalp, it is possible to further accurately measure which portion in the brain surface layer of the subject is activated to what degree. As a result, even when blood flow, or the like, in the scalp largely varies, it is less likely to erroneously estimate emotion, or the like, of the subject.

As described above, as shown in FIG. 8, an example in which a reference signal is acquired by a pair of light-emitting probe and light-receiving probe is described. That is, in the example shown in FIG. 8, one measurement portion for a reference signal is set.

However, when reference signals are measured at many measurement portions, it is possible to further accurately remove noise components.

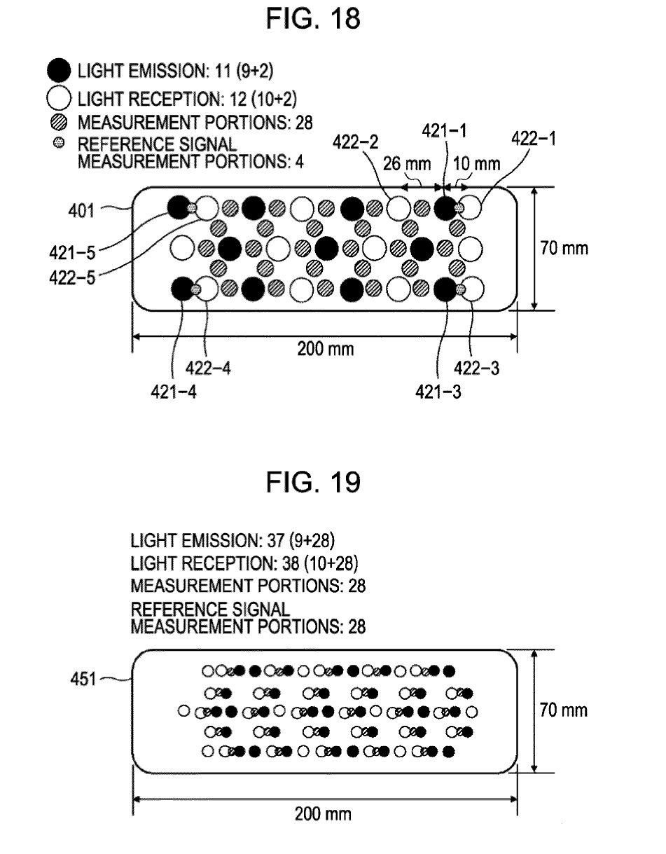

FIG. 18 is a view that shows an example of portions at which light-emitting probes and light-receiving probes are attached when reference signals are acquired at a plurality of measurement portions. In the drawing, as in the case of FIG. 7, black circles in the drawing represent positions at which the light-emitting probes are attached, white circles in the drawing represent positions at which the light-receiving probes are attached, and hatched circles represent optical paths (corresponding to measurement portions). In the example of the drawing, 11 light-emitting probes and 12 light-receiving probes are attached in a region 401 of 70 mm in the vertical direction and 200 mm in the horizontal direction in the drawing on the scalp of the subject, and 28 measurement portions are set in total.

Note that the size of the region 401 in the drawing is an example, and the vertical length and/or horizontal length of the region 401 in the drawing vary depending on the number of measurement portions, or the like.

In the example of FIG. 18, different from the case of FIG. 7, no region corresponding to the region 302 for attaching the light-emitting probe and the light-receiving probe, which acquire a reference signal, is provided.

In addition, in the example of FIG. 18, different from the case of FIG. 7, distances between the light-emitting probes and the light-receiving probes, which are attached at four corners of the substantially rectangular region 401, are shorter than the distances between the other light-emitting probes and light-receiving probes. For example, the distance between the light-emitting probe 421-1 and the light-receiving probe 422-2 is set at 26 mm, whereas the distance between the light-emitting probe 421-1 and the light-receiving probe 422-1 is set at 10 mm. Similarly, the distances between the light-emitting probes 421-3 to 421-5 and the light-receiving probes 422-3 to 422-5 each are also set at 10 mm.

That is, in the example of FIG. 18, four reference signals are acquired by the pair of light-emitting probe 421-1 and light-receiving probe 422-1 and the pairs of light-emitting probes 421-3 to 421-5 and light-receiving probes 422-3 to 422-5. In this example, four reference signals are acquired as described above. Thus, among the light-emitting probes and the light-receiving probes attached in the region 401 for acquiring brain signals, only by reducing the distances between predetermined light-emitting probes and light-receiving probes, it is possible to acquire a desired number of reference signals.

As shown in FIG. 18, when the light-emitting probes and the light-receiving probes are attached, four reference signals may be acquired. Thus, for example, an average of output values of acquired four reference signals may be used as an output value of the reference signal in FIG. 9. By so doing, for example, it is possible to suppress the influence of local changes, or the like, in blood flow in the scalp of the subject 31 on an output value of the reference signal. Thus, it is possible to further accurately remove noise components.

In addition, as shown in FIG. 18, when the light-emitting probes and the light-receiving probes are attached, for example, an output value of a reference signal measured at a position closest to the measurement portion corresponding to each measurement channel may be used as an output value of the reference signal in FIG. 9. By so doing, for example, even when local changes, or the like, in blood flow of the scalp of the subject 31 occur, it is possible to appropriately remove noise components of brain signals of the respective measurement channels.

Alternatively, as shown in FIG. 18, when the light-emitting probes and the light-receiving probes are attached, a reference signal that is most strongly linear to a brain signal of a predetermined measurement channel, from which noise components are removed, may be selected and used from among four reference signals. By so doing, for example, it is possible to further improve reliability of values of coefficients determined by the coefficient determination unit 202.

In addition, as shown in FIG. 18, when four reference signals are acquired, it is possible to obtain four approximate lines on the basis of plotted points described with reference to FIG. 9. For example, it is assumed that, when an output value of a brain signal of a predetermined measurement channel, from which noise components are removed, is plotted on the same graph (graph of FIG. 9) as these four approximate lines, the plotted point is located between the second approximate line and the third approximate line among the four approximate lines. In this case, for example, a value obtained as an average of the second approximate line and the third approximate line may be removed as noise components.

That is, when four different approximate lines having substantially the same slope are obtained in correspondence with four reference signals, an imaginary approximate line corresponding to a position at which the output value of the brain signal of the predetermined measurement channel is plotted may be generated and noise components may be determined from the imaginary approximate line.

That is, as shown in FIG. 18, when the light-emitting probes and the light-receiving probes are attached, it is also applicable that approximate lines corresponding to four reference signals are obtained, and then noise components are calculated on the basis of distances between an output value of a brain signal of a predetermined measurement channel, from which noise components are removed, and those approximate lines.

Note that, for example, when the slopes of four approximate lines are all different, or when four approximate lines are substantially the same, the manner to determine noise components by generating an imaginary approximate line as described above will not be used.

FIG. 19 is a view that shows further another example of positions at which light-emitting probes and light-receiving probes are attached when reference signals are acquired at a plurality of measurement portions. In the drawing, as in the case of FIG. 7, black circles in the drawing represent positions at which the light-emitting probes are attached, white circles in the drawing represent positions at which the light-receiving probes are attached, and hatched circles represent optical paths (corresponding to measurement portions). In the example of the drawing, 37 light-emitting probes and 38 light-receiving probes are attached in a region 451 of 70 mm in the vertical direction and 200 mm in the horizontal direction in the drawing on the scalp of the subject, and 28 measurement portions are set in total.

Note that the size of the region 451 in the drawing is an example, and the vertical length and/or horizontal length of the region 451 in the drawing vary depending on the number of measurement portions, or the like.

In the example of FIG. 19, different from the case of FIG. 7, no region corresponding to the region 302 for attaching the light-emitting probe and the light-receiving probe, which acquire a reference signal, is provided.

In the example of FIG. 19, measurement portions for reference signals are set at positions corresponding to measurement portions of brain signals, and reference signals are acquired at 28 measurement portions. That is, in the example of FIG. 19, hemoglobin concentration in vessels of the brain surface layer is measured, and blood flow in vessels in the scalp outside the vessels of the brain surface layer (immediately above the brain surface layer) is also measured. That is, in the case of FIG. 19, to remove noise components of a brain signal of each measurement channel, reference signals corresponding to the number of measurement channels are respectively acquired at most appropriate positions.

By so doing, it is possible to further appropriately remove noise components.

However, as in the case of FIG. 19, when the light-emitting probes and the light-receiving probes are attached, the number of probes is large. Thus, setting of attachment positions, work for attachment, and the like, are complex. Hence, it is highly likely to require time and cost for obtaining ideal measurement results.

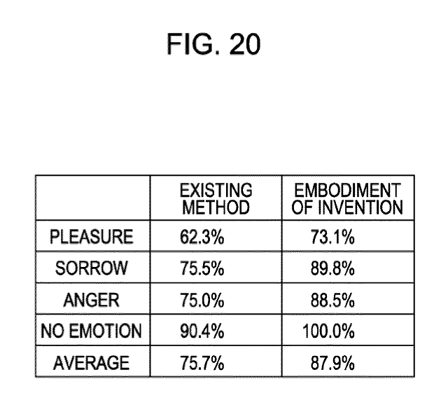

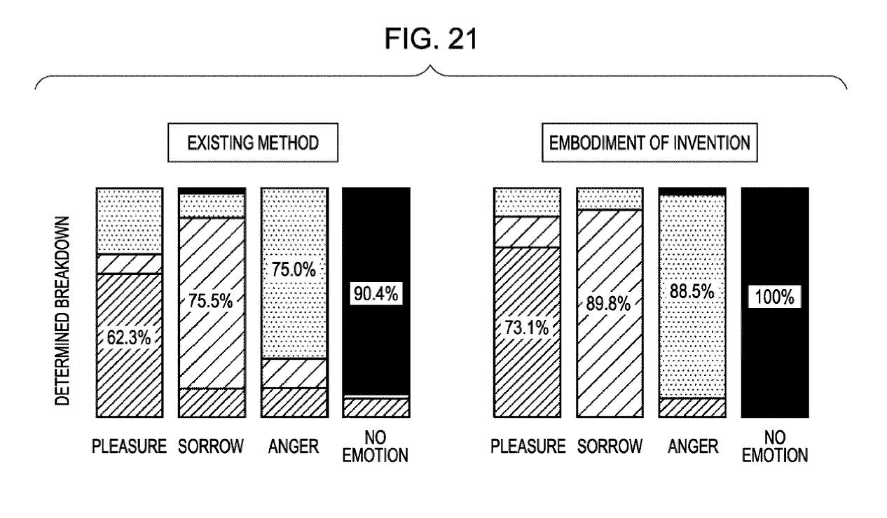

FIG. 20 and FIG. 21 are views for illustrating advantageous effects according to the embodiment of the invention.

FIG. 20 is a table of comparison between percentage of correctness when emotion of the subject is estimated on the basis of brain signals measured through existing fNIRS and percentage of correctness when emotion of the subject is estimated on the basis of brain signals measured using the information processing system 1 according to the embodiment of the invention. This table shows the estimated results of emotion (or feeling) of the subject on the basis of the measured brain signals of the subject in a state where a predetermined stimulus that recalls emotion of “pleasure”, “sorrow” or “anger” is applied to the subject or no specific stimulus is applied to the subject to obtain “no emotion”. It is determined to be correct when emotion estimated on the basis of the measurement results of the brain signals of the subject coincides with emotion actually felt by the subject.

That is, FIG. 20 represents how much emotion of the subject can be correctly estimated using the existing fNIRS or using the information processing system 1 according to the embodiment of the invention without any information about the content of stimulus applied to the subject.

As shown in FIG. 20, percentage of correctness is higher when emotion of the subject is estimated on the basis of brain signals measured using the information processing system 1 according to the embodiment of the invention than the existing art with respect to any emotion of “pleasure”, “sorrow”, “anger” or “no emotion”. In addition, of course, the embodiment of the invention is higher in average percentage of correctness with respect to each emotion.

FIG. 21 is a graph that shows the breakdown of each estimated result in FIG. 20. In the drawing, emotions of “pleasure”, “sorrow”, “anger” and “no emotion” are respectively represented as “pleasure”, “sorrow”, “anger” and “no emotion”.

In FIG. 21, the graph at the left side in the drawing represents the breakdown of the estimated results when emotion of the subject is estimated on the basis of brain signals measured using the existing fNIRS, and the graph at the right side in the drawing represents the breakdown of the estimated results when emotion of the subject is estimated on the basis of brain signals measured using the information processing system 1 according to the embodiment of the invention.

For example, the bar graph corresponding to “pleasure” in the graph at the left side in the drawing represents which is the estimated result of fNIRS, “pleasure”, “sorrow”, “anger” or “no emotion”, when emotion felt by the subject is “pleasure”. In this example, it appears that, when emotion felt by the subject is “pleasure”, 62.3% results of fNIRS are estimated as “pleasure” (that is, percentage of correctness), and many results of fNIRS are erroneously estimated as “anger”.

Similarly, the bar graph corresponding to “pleasure” in the graph at the right side in the drawing represents which is the estimated result of fNIRS, “pleasure”, “sorrow”, “anger” or “no emotion”, when emotion felt by the subject is “pleasure”. Thus, it appears that, when emotion felt by the subject is “pleasure”, 73.1% results of fNIRS (in this case, the information processing system 1 according to the embodiment of the invention) are estimated as “pleasure” (that is, percentage of correctness), and few results of fNIRS are erroneously estimated as “anger”.

Note that the estimated results of emotion of the subject based on brain signals measured using the information processing system 1 according to the embodiment of the invention shown in FIG. 20 and FIG. 21 are estimated results of emotion of the subject based on brain signals measured with the light-emitting probe and the light-receiving probe attached to obtain one reference signal as shown in FIG. 7.

Thus, according to the embodiment of the invention, it is possible to further accurately estimate emotion of the subject.

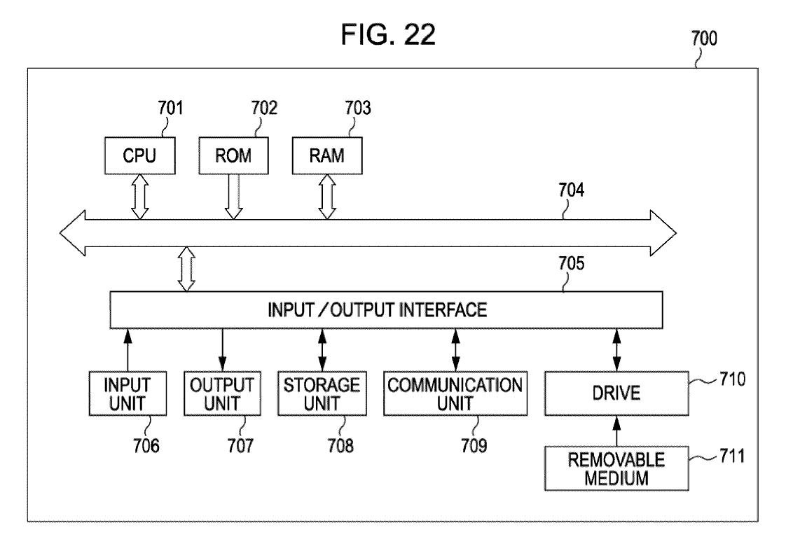

Note that the above described series of processes may be executed by hardware or may be executed by software. When the series of processes are executed by software, programs that constitute the software are installed through a network or from a recording medium onto a computer that is assembled to exclusive hardware or, for example, a general-purpose computer 700, shown in FIG. 22, that is able to execute various functions by installing various programs.

In FIG. 22, a CPU (Central Processing Unit) 701 executes various processes in accordance with programs stored in a ROM (Read Only Memory) 702 or programs loaded onto a RAM (Random Access Memory) 703 from a storage unit 708. The RAM 703 appropriately stores data, or the like, necessary for the CPU 701 to execute various processes.

The CPU 701, the ROM 702 and the RAM 703 are connected via a bus 704. An input/output interface 705 is also connected to the bus 704.

An input unit 706 formed of a keyboard, a mouse, or the like, a display formed of a CRT (Cathode Ray Tube), a LCD (Liquid Crystal display), or the like, an output unit 707 formed of a speaker, the storage unit 708 formed of a hard disk, or the like, and a communication unit 709 formed of a network interface card, such as a modem and a LAN card, are connected to the input/output interface 705. The communication unit 709 executes communication process through a network including the Internet.

A drive 710 is connected to the input/output interface 705 where necessary, a removable medium 711, such as a magnetic disk, an optical disk, a magneto-optical disk or a semiconductor memory, is mounted on the drive 710 where necessary, and then a computer program read from the removable media 711 is installed in the storage unit 708 where necessary.

When the above described series of processes are executed by software, programs that constitute the software are installed through a network, such as the Internet, or from a recording medium formed of the removable medium 711, or the like.

Note that, as shown in FIG. 22, the recording medium is not only formed of, other than the apparatus body, a removable medium 711 which is distributed for providing a program to a user and in which a program is recorded, such as a magnetic disk (including floppy disk (trademark)), an optical disk (including CD-ROM (Compact Disk-Read Only Memory) and DVD (Digital Versatile Disk)), a magneto-optical disk (MD (Mini-Disk) (trademark)) or a semiconductor memory, but also the ROM 702 or the hard disk of the memory unit 708, which are provided for a user in a state of being incorporated in the apparatus body and in which a program is recorded.

Note that in the specification, steps that execute a series of processes include not only processes executed in the written order in time sequence but also processes that are executed in parallel or separately even when the processes are not executed in time sequence.

The present application contains subject matter related to that disclosed in Japanese Priority Patent Application JP 2008-138996 filed in the Japan Patent Office on May 28, 2008, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. An information processing apparatus comprising:
internal information acquisition means for acquiring pieces of information that are acquired by a measuring tool attached to a head of a subject and that indicate changes in blood components at measurement portions inside the head of the subject;
noise removed signal extracting means for removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and
output means for outputting the brain signal, from which the noise components are removed, as a signal indicating an activated state at the corresponding measurement portion of a brain surface layer of the subject.

2. The information processing apparatus according to claim 1, further comprising:
coefficient determination means for determining, on the basis of the reference signal and the brain signal that are acquired in a state where no specific stimulus is applied to the subject, coefficients of a relational expression for obtaining an output value of the brain signal at each measurement portion on the basis of an output value of the reference signal, wherein
the noise removed signal extracting means obtains noise components included in the brain signal on the basis of the determined coefficients to remove the noise components.

3. The information processing apparatus according to claim 1, wherein
the measuring tool is formed of a light-emitting probe that emits near infrared light and a light-receiving probe that receives near infrared light emitted from the light-emitting probe, wherein
the light-emitting probe and the light-receiving probe are attached at a position corresponding to each measurement portion of the head of the subject, and wherein
a distance between the light-emitting probe and the light-receiving probe that are used to generate the reference signal is shorter than a distance between the light-emitting probe and the light-receiving probe for acquiring information used to generate the brain signal.

4. The information processing apparatus according to claim 3, wherein measurement portions for a plurality of the reference signals are set in correspondence with the respective measurement portions of the brain signals.

5. The information processing apparatus according to claim 4, wherein, among the measurement portions of the brain signals, distances between the light-emitting probes and the light-receiving probes at predetermined measurement portions are made short, whereby the predetermined measurement portions serve as the measurement portions for the reference signals.

6. The information processing apparatus according to claim 3, wherein one measurement portion for the reference signal is set at a predetermined position.

7. The information processing apparatus according to claim 6, wherein the measurement portion for the reference signal is set at a position corresponding to a longitudinal fissure of cerebrum of the brain in the head of the subject.

8. The information processing apparatus according to claim 7, wherein the measurement portion for the reference signal is set at a position 20 mm to 30 mm above a glabella of the subject and at a forehead of the subject with no hair.

9. An information processing method comprising the steps of:
acquiring pieces of information that are acquired by a measuring tool attached to a head of a subject and that indicate changes in blood components at measurement portions inside the head of the subject;
removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and
outputting the brain signal, from which the noise components are removed, as a signal indicating an activated state at the corresponding measurement portion of a brain surface layer of the subject.

10. A program for causing a computer to function as an information processing apparatus comprising:
internal information acquisition means for acquiring pieces of information that are acquired by a measuring tool attached to a head of a subject and that indicate changes in blood components at measurement portions inside the head of the subject;
noise removed signal extracting means for removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and
output means for outputting the brain signal, from which the noise components are removed, as a signal indicating an activated state at the corresponding measurement portion of a brain surface layer of the subject.

11. A recording medium in which the program according to claim 10 is recorded.

12. An information processing apparatus comprising:
an internal information acquisition unit for acquiring pieces of information that are acquired by a measuring tool attached to a head of a subject and that indicate changes in blood components at measurement portions inside the head of the subject;
a noise removed signal extracting unit for removing noise components of a brain signal on the basis of a reference signal generated on the basis of information indicating changes in blood components at a predetermined measurement portion among the acquired pieces of information indicating changes in blood components at the measurement portions, wherein the brain signal is generated on the basis of information indicating changes in blood components at the measurement portion different from the predetermined measurement portion; and
an output unit for outputting the brain signal, from which the noise components are removed, as a signal indicating an activated state at the corresponding measurement portion of a brain surface layer of the subject.
